(19) United States
(12) Patent Application Publication
(10) Pub. No.: US 2009/0235932 A1
(43) Pub. Date: Sep. 24, 2009

(54) Respiratory face mask and breathing circuit assembly

(76) Inventor: Ramses Nashed, Tierra Verde, FL (US)

Correspondence Address: Ramses Nashed, 505 First Avenue South, Tierra Verde, FL, 33715, US

(21) Appl. No.: 12/455,048

(22) Filed: May 28, 2009

Related U.S. Application Data
(63) Continuation-in-part of application No. 12/221,379, filed on Aug. 1, 2008.
Application No. 12/221,379 is a continuation-in-part of application No. 11/809,435, filed on Jun. 1, 2007.

(51) Int. Cl. A61M 16/06; A61M 16/12

(52) U.S. Cl. 128/203.29; 128/206.28; 128/206.24

(57) ABSTRACT

A respiratory face mask is provided suitably sized and configured to engage at least the nose portion and mouth portion of a patient's face. Gas from a gas supply and scavenging set normally used in dentistry is operatively coupled to the mask via a breathing circuit to supply fresh gas and evacuate exhaled waste gas. A gas flow compensator in the breathing circuit automatically prevents re-breathing of exhaled gas. Because the mask has a sealing cushion, contamination of the clinical room where the apparatus is being used also is avoided.

### RELATED APPLICATIONS

The present application is a continuation-in-part (CIP) of my prior copending U.S. patent application Ser. No. 12/221,379; filed Aug. 8, 2008, which in turn is a continuation-in-part (CIP) of my copending prior application Ser. No. 11/809,435; filed Jun. 1, 2007 (herein “prior applications”). The aforesaid prior applications hereby are incorporated herein and made part hereof by this reference.

### BACKGROUND OF THE INVENTION

1. Field of the Invention

The present application relates generally to devices for delivering respiratory gas or gases in medical applications, and more particularly, to a new and improved respiratory face mask and breathing circuit assembly that is especially useful in administering gases, such as anesthesia inhalation agents, or oxygen, or air, or mixtures of same, for medical purposes, and as such represents an improvement over the similar device or devices disclosed in my prior applications.

2. Description of the Prior Art

In my prior applications, I fully disclose a disposable anesthesia face mask assembly having a flexible hollow inflatable sealing cushion extending along substantially the entire peripheral edge of the mask shell or body member to prevent anesthesia gas or the like delivered through the gas portal on the mask from escaping and adversely affecting the surgical environment. The unique face mask assembly disclosed in my prior applications represents a significant advance over the prior art because, inter alia, it is the only respiratory or anesthesia mask now known having a pneumatic sealing cushion that extends over and under the chin portion of the patient's face and surprisingly, as a result of this unique arrangement, requires only a relatively simple single headstrap or band for maintaining the mask in a stable position even during long duration surgical procedures. Hence, the mask and headstrap assembly of my prior applications are ideally suited for use during so-called “sedation” anesthesia procedures where the patient breathes on its own.

A well-known safe and effective sedation anesthetic in gas inhalation form is nitrous oxide (N₂O). This compound, frequently called “laughing gas” or “happy gas,” has been used medically for many years usually as an analgesic or “light sedative” to ease pain associated with trauma, childbirth or heart attacks, for example, or as a carrier for more potent anesthesia gases (e.g. Desflurane, Sevoflurane). Indeed, nitrous oxide is so safe, easy to handle, and readily available relatively inexpensively, it has been used as an anesthetic in dentistry since the 1800s. When used by a dentist, nitrous oxide is typically administered through a demand-valve or continuous inhaler placed over the patient's nose so that the patient's mouth can be worked on while the patient continues to inhale the gas. A full face-mask therefore cannot be used in dentistry, and to avoid build-up of exhaled or waste nitrous oxide, which can pose an intoxicating and prolonged-exposure hazard to the clinic staff in the room where the procedure is being conducted, a continuous-flow-fresh-air ventilation system or nitrous-scavenging system must be used.

In “Case Series of Nurse-Administered Nitrous Oxide for Urinary Catheterization in Children,” Anesthesia & Analgesia 2007, 104:876-879, Judith L. Zier, M D. et al, the authors reported the use N₂O to “sedate” children undergoing a painful procedure in a hospital setting. Administration of the nitrous was carried out using commercially available “dental” equipment to supply the N₂O gas and to scavenge exhaled waste (through a nose mask). Thus, the authors reported that a “comfortable seal could be maintained . . . over the nose of the older child” (emphasis added). Nitrous oxide is only minimally metabolized in the human body. Accordingly, unless provision is made to collect or otherwise scavenge the breath of a patient exhaled through the mouth, build up of exhaled N₂O waste gas in the hospital environment using such “dental equipment” cannot reasonably be avoided.

In order to overcome the foregoing disadvantages, the present application discloses a new and improved respiratory face mask and breathing circuit assembly adapted to be used with commercially available nitrous oxide gas handling equipment commonly and heretofore used in “dentistry.” When so employed, the respiratory face mask and breathing circuit assembly of the present invention completely avoids contaminating the surrounding environment with built-up waste N₂O gas thereby facilitating safe and effective “combined” use of such equipment for “sedation” purposes in a hospital or other clinical environment.

### BRIEF SUMMARY OF THE INVENTION

The present invention, briefly described, essentially comprises the respiratory face mask of my prior applications improved by a supplemental unique breathing circuit enabling the face mask to be seamlessly coupled to a conventional “dental” nitrous gas set-up having a fresh-gas-supply module and a vacuum or scavenging module. The unique breathing circuit includes at one end a plug for insertion within the main gas flow passage of the mask. A fresh-gas, flexible supply tube is joined to the breathing circuit plug at one end and is adapted to be coupled to the outlet nozzle on the fresh gas supply module of the “dental” set-up. At the other or opposed end of the breathing circuit, a collapsible, flexible gas reservoir bag is suitably provided. The bag's distal end is joined to one end of a gas flow compensator which, in turn, has extending from its other opposed end a flexible tube suitably adapted to be coupled to the vacuum module of the “dental” set-up. When so arranged, the face mask, breathing circuit including the gas flow compensator of the present invention permits fresh gas supplied from the “dental” set-up to be inhaled continuously by a patient being “sedated” and any and all exhaled waste gas whether expelled through the patient's nose or mouth is safely scavenged through the vacuum module of the “dental” setup thereby avoiding re-breathing of exhaled gas by the patient and any build-up of waste N₂O gas in the surrounding environment.

There has thus been described, rather broadly, several important features of the invention in order that the detailed description thereof that follows may be better understood and in order that the present contribution to the art may be better appreciated. There are, of course, additional features of the invention that will be described hereinafter and which will form the subject matter of the claims attached.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved respiratory face mask and breathing circuit assembly which has all of the advantages of the prior art and none of the disadvantages.

It is another object of the present invention to provide a new and improved face mask and breathing circuit that is especially suitable for use in administering anesthesia inhalation gas or gases to a patient continuously without causing any exhaled waste gas to build-up in the surrounding area.

It is further object of the present invention to provide a new and improved face mask and breathing circuit assembly which is of durable and reliable constructions.

An even further object of the present invention is to provide a new and improved respiratory face mask and breathing circuit assembly which is susceptible of a low cost of manufacture with regard to both materials and labor, and which accordingly is then susceptible of low prices of sale, thereby making such a face mask and breathing circuit assembly economically available to the medical community.

Even still another object of the present invention is to provide a new and improved respiratory face mask and breathing circuit assembly that may be used on a patient before, during or after surgery in a safe and efficient manner.

It is yet another object of the present invention to provide a new and improved respiratory face mask and breathing circuit assembly where the breathing circuit is adapted to be coupled to the fresh gas supply module of a nitrous oxide “dental set” and further includes means for connecting the breathing circuit to the vacuum module of the same “dental set.”

Still yet another object of the present invention is to provide a new and improved respiratory face mask and breathing circuit assembly where the breathing circuit is adapted to be coupled to the fresh gas supply module of a nitrous oxide “dental set” and further includes means for automatically preventing re-breathing of waste gas exhaled into said breathing circuit.

Yet still yet another object of the present invention is to provide a new and improved respiratory face mask and breathing circuit assembly where the breathing circuit is adapted to be coupled to the fresh gas supply module of a nitrous oxide “dental set” and further includes a gas flow compensator for substantially avoiding re-breathing of gas exhaled into the breathing circuit by the patient.

Another object of the present invention is to provide a new and improved respiratory face mask characterized by at least two primary components, namely a cup-like shell or receptacle member terminating at its peripheral edge in a peripheral flange, and an annular substantially donut-shaped hollow inflatable cushion or sealing member affixed or otherwise permanently attached to the flange along substantially the flange's entire peripheral extent, in combination with a breathing circuit adapted to be coupled between the cup-like shell member and a device for supplying anesthesia gas normally used in dentistry.

Even still yet another object of the present invention is to provide a disposable anesthesia face mask and breathing circuit assembly ideally suited for use during “sedation” anesthesia procedures where the patient is self-breathing.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a schematic perspective view of the respiratory face mask and breathing circuit assembly of the present invention where the mask is depicted fitted to a patient and being connected to the fresh gas supply module and vacuum module, respectively, of an anesthesia gas supply and scavenging device.

FIG. 2 is an enlarged cross-sectional view in elevation of a portion of the respiratory face mask and breathing circuit assembly of FIG. 1 taken along line 2-2 of FIG. 3 (i.e. element 62).

FIG. 3 is a cross-sectional view taken along line 3-3 in FIG. 2.

FIG. 4 is an assembly view of the various parts making up the respiratory face mask and breathing circuit assembly of the present invention.

### DESCRIPTION OF THE PREFERRED EMBODIMENT

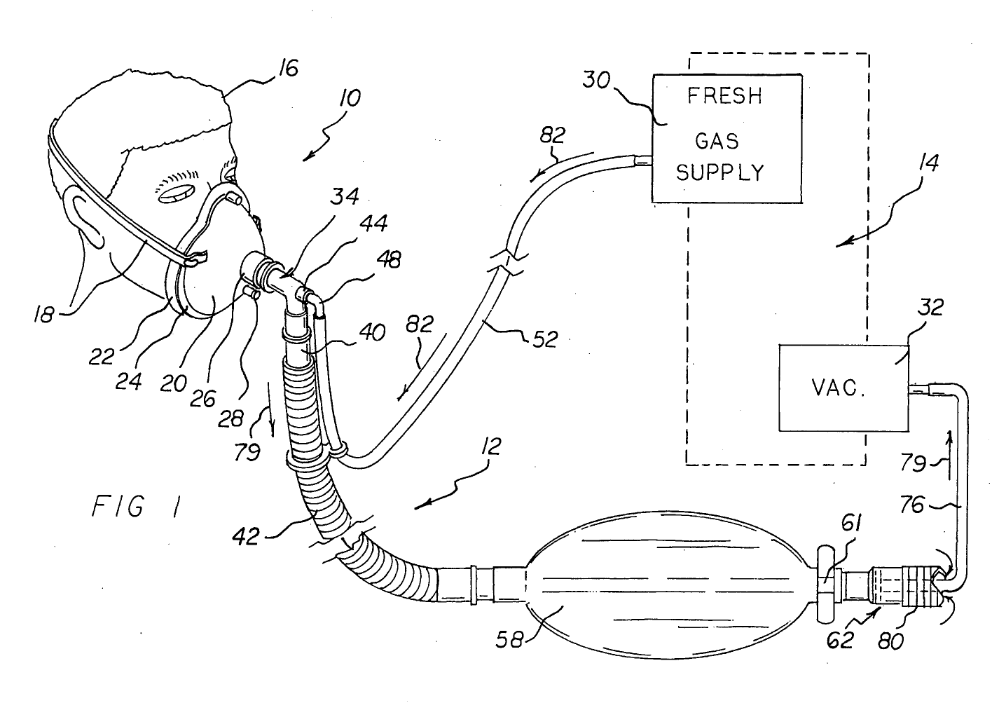

With reference now to FIGS. 1-4 of the Drawings, there is shown a preferred embodiment of the new and improved respiratory face mask and breathing circuit assembly of the present invention generally comprising a face mask 10, a breathing circuit 12, and a gas supply and scavenging device 14.

As schematically depicted in FIG. 1, face mask 10 is fitted on the face of a patient 16 via a headstrap 18 (preferably elastic) removably attachable to the face mask via anchors or the like on the opposed edges thereof. The headstrap helps to stabilize the mask on the face and head of a person, preferably before, during and/or after surgery. Many of the details of construction of the face mask 10 and headstrap 18 are outside the scope of the present invention. For a more detailed understanding of same, the interested reader is directed to my prior applications (incorporated herein by reference). Suffice it to say for purposes of fully understanding the present invention, face mask 10 preferably is similar to that fully disclosed in my prior applications and therefore, comprises a cup-like shell or receptacle member 20 and a flexible pneumatic (inflatable) cushion member 22. The flexible pneumatic inflatable cushion member 22 defines a generally donut-shaped sealing member extending annually along the entire extent of a peripheral rim or flange 24 defined by the shell member 20.

As further disclosed in my prior applications, the crown portion of shell member 20 has a first cylindrical hollow inlet member or port 26 suitably sized to removably receive in snug sliding (and sealing) engagement therein the nipple of a conventional breathing circuit and to define a passageway such that anesthesia gas or other gases are adapted to pass from a source through the breathing circuit and the passageway into the interior of shell member 20. The breathing circuit also is adapted to receive returned or exhaust gas or gases through the same passageway and convey such exhausted gas or gases to a conventional gas evacuation or scavenging apparatus such as is provided on a standard anesthesia machine used in surgery rooms. Shell member 20 also may include a second cylindrical hollow outlet member or port 28 for connection to a length of flexible tube (not shown) the other or distal end of which is adapted suitably to be connected to a conventional CO₂ monitoring device or capnograph for sensing end-tidal CO₂ of a breathing patient.

As further disclosed in my prior applications, when the face mask 10 is applied to the face of a patient, the top portion of the mask receives and covers the nose portion of the patient, the intermediate portion of mask covers the mouth portion of the patient, and the bottom or widest portion of the mask receives and engages the underside of the chin and jaw portion of the patient's face with the pneumatic cushion member 22 effectively serving to seal the periphery of the mask relative to the patient's face and preventing escape of anesthesia gas or other gas into the surrounding environment notwithstanding the irregular contour of the patient's face. As further disclosed in my prior applications, the foregoing engagement between the underside of the chin or jaw portion of the patient with the bottom or widest transverse edge portion of the mask prevents displacement of the mask toward the ocular area, helps to keep the patient's mouth closed, and securely stabilizes the mask assembly on the face when the elastic headstrap is used thereby freeing both hands of attending anesthesia personnel for use elsewhere. It will be appreciated in connection with carrying out the present invention, that the face mask 10, depicted in FIG. 1, can be fitted to the face and head of a patient in the foregoing manner, or in the manner described in my prior applications.

In accordance with the present invention, and as schematically depicted in FIG. 1, a uniquely modified breathing circuit 12 is employed to deliver anesthesia gases or the like originating from gas supply and scavenging device 14 to face mask 10. Device 14 is of the well-known type commonly used in the practice of dentistry and comprises inter alia a fresh gas supply module 30 and a vacuum module 32 for scavenging waste gas or exhaled gas. By way of example, such devices are commercially available from Porter Instrument, Hatfield, Pa. 19440 and now are in widespread use. Thus, as is well known, the gas supply module 30 in such “dental” devices is adapted to supply both nitrous oxide and oxygen and has valve controls for adjusting the flow of each to produce a mixture of any desired concentration. The scavenging module 32 provides a source of negative pressure (vacuum) and can be wall mounted to provide a convenient outlet. It will be understood that the constructional details of these devices are conventional and outside the scope of the present invention.

Modified breathing circuit 12 includes a Y-connector 34 having a first tubular plug or nipple 36 suitably sized for mating engagement within the first cylindrical inlet member or port 26 on face mask 10. Y-connector 34 also features a second tubular branch or nipple 38 for mating with a first open cylindrical end 40 of flexible corrugated tube 42 and a third tubular branch or receptacle 44 for mating with a first tubular end or nipple 46 of elbow connector 48. The second or opposed end 50 of elbow connector 48 is adapted to be connected to a flexible “fresh gas” supply tube 52 which in turn is connected to the output of the gas supply module 30 of “dental” gas supply and scavenging device 14. (FIG. 1).

Flexible corrugated tubing 42 terminates in a second open end 54 which in turn is adapted to be matingly engaged with the first cylindrical open end 56 of flexible collapsible reservoir bag 58. The opposed second cylindrical end of reservoir bag 58 terminates in a cylindrical plug 60 having a rotatable gate valve therein (not shown) controlled by handle 61. Rotation of handle 61 in either direction about the central longitudinal axis of the plug 60 varies the size of a suitable opening provided in the distal end of plug 60 between a minimum and a maximum extent, respectively. The purpose of the gate valve is to adjust the flow of exhaled waste gas through the reservoir bag 58 somewhat if desired as will be made more evident below.

Thus far breathing circuit 12 is similar to a prior art Modified Jackson Rees (MJR) breathing circuit used in General Anesthesia applications. In order to adapt breathing circuit 12 for purposes of the present invention, the cylindrical plug 60 located on the second end of reservoir bag 58 is operatively coupled to a gas flow compensator 62 the purpose of which is to automatically assure that any waste gas exhaled into the breathing circuit will not be re-breathed by the patient, or escape into the surrounding environment, but rather will safely be evacuated via the vacuum module 34 of gas supply and scavenging device 14. Gas flow compensator 62 generally is tubular in shape and has a first end defining a cylindrical receptacle 64 for receivingly mating with plug 60. The opposed second end of compensator 62 defines a cylindrical opening 66 exposed to the surrounding environment.

Alternatively, gas flow compensator 62 may advantageously be integrated with plug 60 to form a one-piece or unitary construction. In such a case, the gate valve and its handle 61 is obviated and may be may dispensed with if desired.

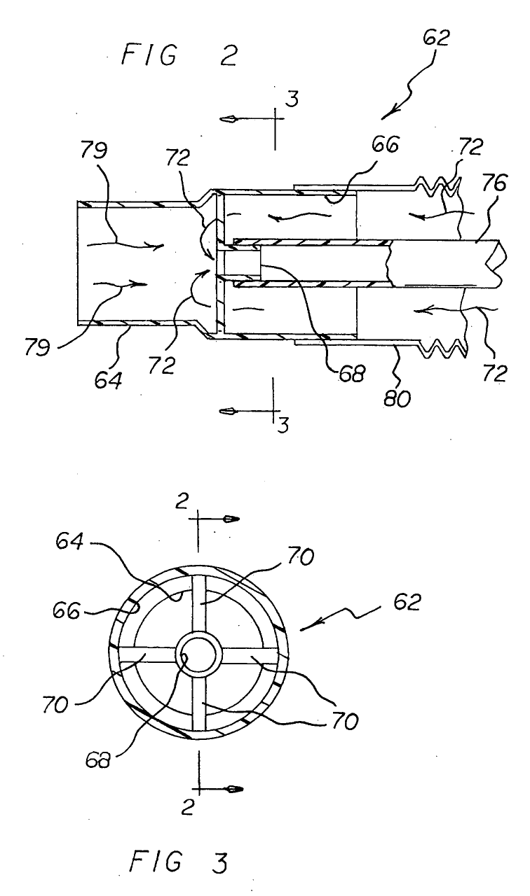

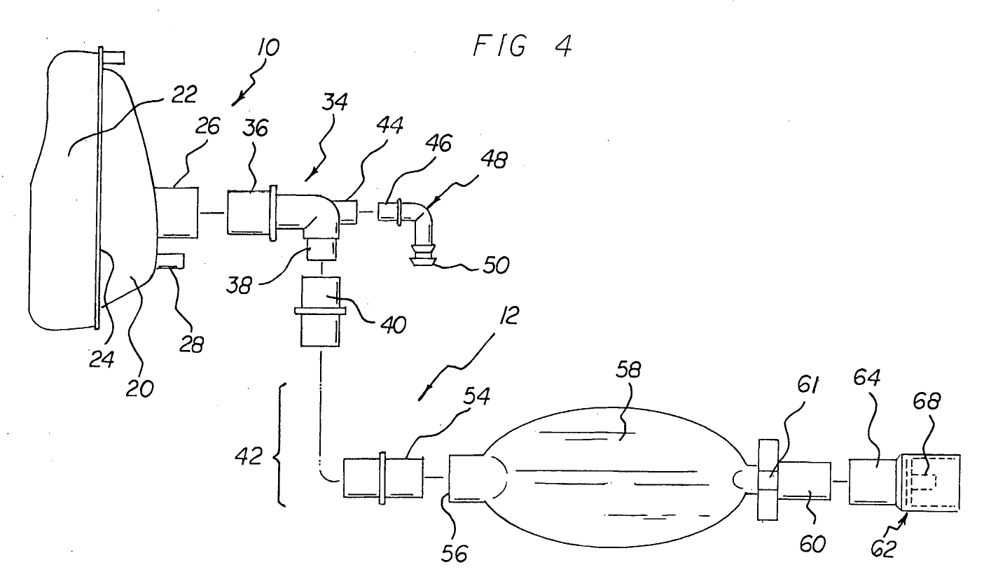

Mounted interiorly of compensator 62 substantially medially of the first and second opposed ends thereof and coaxially therewith is a nozzle 68 of reduced diameter relative to both receptacle 64 and opening 66 substantially as shown in FIGS. 2 and 3. Nozzle 68 preferably is supported at its leftmost end (FIGS. 2 and 3) by a series of four radial struts 70 which serve as a spider suspending the nozzle in its coaxial orientation, but which allow a quantity of air from the environment to pass through opening 66 from the right to the left, thence through the openings in the spider as indicated by arrows 72, before entering nozzle 68 through the latter's leftmost open side as viewed in FIG. 2. A length of flexible tubing 76 is suitably securely affixed to nozzle 68 (FIG. 2) proximal to struts 70 and extends coaxially therefrom out through open end 66 of the compensator 62 ultimately being connected at its other opposed distal end to vacuum module 32 of “dental” device 14. It will be noted that the length or axial extent of nozzle 68 is not critical, and it suffices if this part has an axial extent or length sufficient to provide a firm gripping surface for the end of tube 76 attached thereto.

By the foregoing arrangement of parts, it will be appreciated that any exhaled waste gas entering receptacle 64 from reservoir bag 58 (arrows 79, FIG. 2) as well as a quantity of outside air entering compensator 62 (arrows 72) will be drawn by the vacuum in flexible tube 76 through the left side of nozzle 68 and scavenged by vacuum module 32 of device 14. Compensator 62 thus automatically compensates for any dead space in the breathing circuit which might cause exhaled waste gas in the reservoir bag 58 or in the flexible corrugated tubing 42 to stagnate therein and be re-breathed by a patient. Because the face mask 10 has a sealing cushion, leakage of any gas whatsoever into the surrounding environment also is avoided.

Optionally, a cylindrical extension sleeve 80 can be attached to the rightmost side of compensator 62 to increase the effective axial extent of the compensator and thereby form a supplemental reservoir for any exhaled waste gas that may travel along a path from bag 58 through plug 60 and pass beyond the leftmost side of nozzle 68. Further tweaking of the flow of gases through the gas flow compensator 62 may optionally be attempted by adjusting the position of the handle 61 of the gate valve on the plug 60, but generally this valve should be placed in the fully “open” position during operation of the disclosed apparatus. Here too, if desired, extension tube 80 and the rightmost end of compensator 62 may be formed of a unitary one-piece construction.

In using the apparatus of the invention, and as schematically shown in FIG. 1, it will be seen that fresh gas is adapted to be supplied from supply module 30 on “dental” device 14 to the patient through supply tube 52, Y-connector 34, and mask inlet port 26 (arrow 82). Waste or exhaled gas flows into corrugated tube 42 and reservoir bag 58 under the influence of the vacuum module 32 applying a negative pressure in tube 76 (arrows 79). All that is necessary after fitting mask 10 to the face of a patient is to flush (expand) the bag 58 with a fresh supply of gas from supply module 30 of device 14 through supply tube 52 (using the “flush” button on the fresh gas supply module), attach the breathing circuit Y-connector 34 to the mask inlet, and connect the flexible vacuum tube 76 from the gas flow compensator 62 to the outlet of vacuum module 32. The patient then will breath easily through mask 10 and in so doing draw fresh gas from tube 42 and the flushed reservoir bag 52, collapsing the bag. When the patient exhales, the waste gas will fill the bag (and corrugated tube 42) and be immediately drawn through the gas flow compensator 62 via the negative pressure afforded by vacuum tube 76. At the same time, the negative pressure in tube 76 will cause fresh gas from supply tube 52 to fill corrugated tube 42 and reservoir bag 58 so as the patient breathes again, the patient avoids re-breathing any waste or exhaled gas.

From the foregoing description, it should be evident that the apparatus of the present invention solves the vexing problem of providing a face mask for administering anesthesia gases or other gases employing the relatively portable and inexpensive gas supply and scavenging equipment heretofore used only in the practice of dentistry. By using the face mask of the present invention, with its excellent sealing capability, and the modified breathing circuit according to the present invention, in conjunction with such “dental” devices, re-breathing of waste gas by the patient and build-up of waste gas in the clinical room is avoided. As a result, “sedation” anesthesia employing nitrous oxide may now be practiced more readily in hospitals, surgical centers and doctor's offices in a safe and effective manner thereby leading to increased patient satisfaction and reduced medical costs.

Many variations and modifications to the foregoing will be readily apparent and obvious to one skilled in the art, and all equivalent relationships to those illustrated in the drawings and described in the specification are intended to be encompassed by the present invention. Therefore, it is not desired to limit the invention to the exact construction and operation shown and described, and accordingly, all suitable modifications and equivalents may be resorted to, falling within the scope of the invention as defined in the annexed claims.

The components of the respiratory face mask and breathing circuit assembly of the invention can be made from inexpensive and durable plastic or metal materials.

As to the manner of usage and operation of the instant invention, the same is apparent from the above disclosure, and accordingly, no further discussion relative to the manner of usage and operation need be provided.

Finally, it will be appreciated that the purpose of the annexed Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. Accordingly, the Abstract is neither intended to define the invention or the application, which only is measured by the claims, nor is it intended to be limiting as to the scope of the invention in any way.

What is claimed as being new and desired to be protected by LETTTERS PATENT of the United States is as follows:

1. Apparatus for administering gas to a person in a clinical environment, comprising:
a face mask, said face mask being suitably sized and configured to engage at least the nose portion and mouth portion of said person's face, said face mask further including a mask body and a sealing cushion adapted to be interposed between said mask body and said at least nose portion and said at least mouth portion of said person's face when said face mask engages said person's face,
a breathing circuit adapted to be operatively coupled to said face mask for delivering fresh gas to said mask body and for scavenging exhaled gas received from said mask body when said face mask engages said person's face,
wherein said breathing circuit includes means for automatically preventing gas exhaled into said breathing circuit from being re-breathed by said person.

2. The apparatus of claim 1 in combination with a device for supplying fresh gas to said mask body and for scavenging exhaled gas received from said mask body.

3. The apparatus of claim 1 wherein said supplied gas is nitrous oxide.

4. The apparatus of claim 1 wherein said supplied gas is nitrous oxide, or oxygen, or mixtures of same.

5. The apparatus of claim 1 wherein said face mask includes a further portion for engaging the underside of the person's chin when said face mask engages the person's face and said sealing cushion includes a further portion interposed between said person's chin and said mask body when said face mask engages said person's face.

6. The apparatus of claim 1 wherein said face mask and said breathing circuit are disposable.

7. The apparatus of claim 2 wherein said breathing circuit includes a first tube adapted to receive exhaled gas from said mask body and a gas reservoir attached to said tube and in communication therewith,
said breathing circuit further including a second tube adapted to supply fresh gas to said mask body,
wherein said means for automatically preventing gas exhaled into said breathing circuit from being re-breathed by said person comprises a portion of said breathing circuit having a first end and a second end,
said first end of said portion being adapted to be coupled to said gas reservoir, and said second end of said portion being adapted to be operatively coupled to said device for supplying fresh gas to said mask body and for scavenging exhaled gas received from said mask body.

8. The apparatus of claim 7 wherein said gas reservoir is a flexible collapsible bag.

9. The apparatus of claim 7 wherein said portion of said breathing circuit having a first end and a second end comprises a first outer tube having a first diameter and a second inner tube having a second diameter, said first diameter being greater than said second diameter.

10. The apparatus of claim 7 wherein said second inner tube is a nozzle coaxially supported within said first outer tube and said nozzle has a first end and an opposed second end, said opposed second end of said nozzle being coupled to a vacuum tube, said vacuum tube being adapted to be operatively coupled to said device for supplying fresh gas to said mask body and for scavenging exhaled gas received from said mask body, and wherein said first end of said nozzle is adapted to be in communication with said gas reservoir through said first end of said portion of said breathing circuit having a first end and a second end.

11. The apparatus of claim 2 wherein said device for supplying fresh gas to said mask body and for scavenging exhaled gas received from said mask body is a dental set normally used in the practice of dentistry.

12. The apparatus of claim 10 wherein said device for supplying fresh gas to said mask body and for scavenging exhaled gas received from said mask body is a dental set normally used in the practice of dentistry, said device further including a wall-mounted outlet for a vacuum line.

13. The apparatus of claim 1 wherein said breathing circuit includes a first tube adapted to receive exhaled gas from said mask body and a gas reservoir attached to said tube and in communication therewith,
said breathing circuit further including a second tube adapted to supply fresh gas to said mask body,
said means for automatically preventing gas exhaled into said breathing circuit from being re-breathed by said person comprising a tube having a first end and a second end,
said first end of said tube being operatively coupled to said gas reservoir, and said second end of said tube adapted to be operatively coupled to a source of negative pressure and wherein said tube comprises a first outer tube having a first diameter and a second inner tube having a second diameter, said first diameter being greater than said second diameter,
wherein said second inner tube defines a nozzle coaxially supported within said first outer tube and said nozzle has a first end and an opposed second end, said opposed second end of said nozzle being adapted to be operatively coupled to said source of negative pressure, and wherein
said first end of said nozzle is adapted to be in communication with said gas reservoir through said first end of said tube.

14. The apparatus of claim 13 in combination with a device for supplying fresh gas to said mask body and for scavenging exhaled gas received from said mask body and wherein said device is a dental set normally used in the practice of dentistry, said device further including a controllable supply source of fresh gas, and wherein said second tube for supplying fresh gas to said mask body is adapted to be connected to said controllable supply source of fresh gas.

15. The apparatus of claim 14 wherein said mask body includes an inlet port and said breathing circuit includes a Y-connector, said Y-connector having at least first and a second branches, said at least first branch of said Y-connector being adapted to be operatively coupled to said mask body inlet port, said at least second branch being adapted to be operatively coupled to said second branch of said Y-connector being adapted to be operatively coupled to said second tube for supplying fresh gas to said mask body.

16. The apparatus of claim 15 wherein said Y-connector further includes a third branch, said third branch adapted to be operatively coupled to said first tube for receiving exhaled gas from said mask body and for delivering said exhaled gas to said gas reservoir attached to said tube and in communication therewith.

17. The method of administering sedation anesthesia to a patient comprising the following steps:
a. providing a respiratory face mask having a donut-shaped peripheral sealing cushion;
b. fitting said face mask to the face of a patient such that said face mask sealing cushion extends underneath the patient's chin and engages the nose portion and mouth portion of the patient's face;
c. providing a breathing circuit including means for automatically preventing re-breathing of exhaled gas by said patient;
d. attaching said breathing circuit to said face mask; and
e. applying at least one anesthesia gas to said patient via said fitted face mask through said breathing circuit.

18. The method of claim 17 wherein step (e) comprises the further step of supplying at least one anesthesia gas comprises supplying a mixture of nitrous oxide and oxygen.
